(19) United States
(12) Patent Application Publication
(10) Pub. No.: US 2025/0213688 A1
(43) Pub. Date: Jul. 3, 2025

(54) METHODS AND COMPOSITIONS FOR LOCALIZED PRODUCTION AND DELIVERY OF BIOLOGICAL MOLECULES

(71) Applicant: Iyuda Therapeutics, Inc., Owings Mills, MD (US)

(72) Inventor: Robert E. TENNANT, Owings Mills, MD (US)

(21) Appl. No.: 19/084,250

(22) Filed: Mar. 19, 2025

Related U.S. Application Data
(63) Continuation of application No. PCT/US2023/075306, filed on Sep. 28, 2023.
(60) Provisional application No. 63/377,695, filed on Sep. 29, 2022.

(51) Int. Cl. A61K 40/11; A61K 31/675; A61K 31/7056; A61K 31/7076; A61K 40/35; A61K 40/42; A61K 45/06; A61P 35/00; C12N 5/0783

(52) U.S. Cl. CPC A61K 40/11; A61K 31/675; A61K 31/7056; A61K 31/7076; A61K 40/35; A61K 40/4234; A61K 40/4235; A61K 45/06; A61P 35/00; C12N 5/0636; C12N 2510/00

(57) ABSTRACT

This disclosure relates to leukocyte compositions that comprise immune cells that are modified for enhanced in vivo anti-tumor activity. The leukocyte compositions are enriched for CD4+ Th1 cells or other differentiated states and optionally depleted of CD8+ T cells.

The present application claims the benefit of U.S. Provisional Application No. 63/377,695, filed on Sep. 29, 2022, which is incorporated herein by reference in its entirety.

### 1. BACKGROUND

Immunotherapy has emerged as a promising modality for the treatment of a variety of diseases, including cancer, infectious disease, and autoimmune disease. The goal of immunotherapy is to harness the immune system to achieve a therapeutic end. For example, the goal of cancer immunotherapy is to achieve the selective destruction of cancer cells while leaving normal tissues unharmed.

The effective harnessing of the immune system requires the coordination of numerous components—for example, T cells, macrophages, and dendritic cells—in order to achieve homeostasis or a therapeutic end. This coordination is accomplished through various mechanisms which rely on molecular interactions at the cell surface or via secreted molecules like cytokines or chemokines.

Other systems in the body also utilize secreted molecules, for example, hormones, to maintain homeostasis and to regulate and coordinate the activities of different tissues.

Allogeneic T cell therapy is a form of immunotherapy in which lymphocytes are collected from the peripheral blood or bone marrow of healthy donors and transfused into a patient. This therapy is typically given in the form of allogeneic stem cell transplantation, in which the patient receives highly immunosuppressive conditioning followed by an infusion of a stem cell graft containing unselected populations of mature T cells. The goal of allogeneic stem cell transplantation is to obtain sustained engraftment of the donor cells, but it is associated with significant toxicities including graft-versus-host disease (GVHD), an immunologic attack on normal tissues. International Application No. PCT/US2013/032129 describes an approach termed non-engrafting donor lymphocyte infusion, in which a patient is treated with lymphodepleting chemotherapy followed by a transfusion of peripheral blood cells, depleted of CD8+ cells, from a healthy, partially or fully human leukocyte antigen (HLA)-mismatched donor. The proposed therapeutic mechanism of mismatched, CD8-depleted donor lymphocyte infusion (DLI) is that alloreactive donor CD4+ T cells secrete biological molecules (cytokines) and deliver signals through recipient antigen-presenting cells (APCs) to enhance the immunologic environment, enhance the activation and polarization of immune effector cells, and reverse exhaustion in recipient-derived, tumor-specific CD8+ T cells. While this therapy can induce anti-tumor responses against hematologic malignancies, its efficacy against solid tumors is limited, possibly by the reversion of tumor-specific CD8+ T cells to an exhausted state.

As such, in cancer there continues to be an unmet need for methods that can efficiently and sustainably awaken exhausted T cells and induce regression of tumors while reducing the risk of sustained engraftment and graft-versus-host disease. Furthermore, there are numerous other diseases in which manipulation of the immune and/or other systems could enhance treatment of the condition.

### 2. SUMMARY

The disclosure relates to a pharmaceutical composition comprising a plurality of isolated leukocytes obtained from a subject. The leukocytes are modified to increase the expression of IL-2, FLT3L, IFN gamma, IFN alpha, IFN beta, IL-12, TNF alpha, IL-1 beta, IL-6, CSF2, IL-4, IL-5, IL-10, IL-13, IL-21, TGF-β, or combinations thereof.

The disclosure also relates to a pharmaceutical composition comprising a plurality of isolated leukocytes that are obtained from a donor subject and are mismatched to a recipient subject for at least one human leukocyte antigen (HLA) Class II allele in the donor versus recipient (graft-versus-host) direction relative to the recipient subject. The leukocytes are depleted of CD8+ T cells by about 10-fold or greater relative to un-depleted leukocytes. The leukocytes are modified to increase the expression of IL-2, FLT3L, IFN gamma, CSF2, IL-4, IL-5, IL-10, IL-13, IL-21, TGF-β, IFN gamma, IFN alpha, IFN beta, IL-12, TNF alpha, IL-1 beta, IL-6, or combinations thereof.

The disclosure further relates to a pharmaceutical composition comprising a plurality of isolated leukocytes obtained from an allogeneic donor subject, wherein the donor CD4+ T cells have been stimulated in vivo or ex vivo by an antigen present in a recipient subject, and the donor subject comprises at least one HLA Class II allele match relative to the recipient. The leukocytes are depleted of CD8+ T cells by about 10-fold or greater relative to un-depleted leukocytes. The leukocytes are modified to increase the expression of IL-2, IFN gamma, IFN alpha, IFN beta, IL-12, TNF alpha, IL-1 beta, IL-6, FLT3L, IFN gamma, CSF2, IL-4, IL-5, IL-10, IL-13, IL-21, TGF-β or combinations thereof.

The disclosure relates to a pharmaceutical composition comprising a plurality of isolated leukocytes that are obtained from a donor subject and (i) are mismatched to a recipient subject for at least one HLA Class II allele mismatch in the donor versus recipient (graft-versus-host) direction relative to the recipient subject and (ii) the donor CD4+ T cells have been stimulated in vivo or ex vivo by an antigen present in a recipient subject, and the donor subject comprises at least one human leukocyte HLA Class II allele match relative to the recipient. The leukocytes are depleted of CD8+ T cells by about 10-fold or greater relative to un-depleted leukocytes. A portion of the CD4+ T cells are modified to increase the activity of IL-2, IFN gamma, IFN alpha, IFN beta, IL-12, TNF alpha, IL-1 beta, IL-6, FLT3L, IFN gamma, CSF2, IL-4, IL-5, IL-10, IL-13, IL-21, TGF-β or combinations thereof.

At least a portion of the T cells can be differentiated to Th1 or T follicular helper (Tfh) CD4+ T cells. T cells can be biased toward Th1 CD4+ T cell differentiation by increasing the expression of IL-2, IFN gamma, IFN alpha, IFN beta, IL-12, TNF alpha, IL-1 beta, IL-6, FLT3L, IFN gamma, CSF2, IL-4, IL-5, IL-10, IL-13, IL-21, TGF-β or combinations thereof. The expression of IL-2, IFN gamma, IFN alpha, IFN beta, IL-12, TNF alpha, IL-1 beta, IL-6, FLT3L, IFN gamma, CSF2, IL-4, IL-5, IL-10, IL-13, IL-21, TGF-β or combinations thereof can be increased with a pharmacological agent or by genetic modification. Expression of IL-2, IFN gamma, IFN alpha, IFN beta, IL-12, TNF alpha, IL-1 beta, IL-6, FLT3L, IFN gamma, CSF2, IL-4, IL-5, IL-10, IL-13, or IL-21 can be increased by modifying the IL-2 gene, the FLT3L gene, the IFN gamma gene, the CSF2 gene, the IL-4 gene, the IL-5 gene, the IL-10 gene, the IL-13 gene, the IL-21 gene, or the TGF-β gene. The IL-2 gene, the FLT3L gene, the IFN gamma gene, the CSF2 gene, the IL-4 gene, the IL-5 gene, the IL-10 gene, the IL-13 gene, the IL-21 gene, the IFN gamma gene, the IFN alpha gene, the IFN beta gene, the IL-12 gene, the TNF alpha gene, the IL-1 beta gene, the IL-6 gene, or the TGF-β gene can be modified using CRISPR, TALEN, base editing, prime editing, or PASTE.

Differentiation of the T cells into T regulatory cells can be attenuated with a pharmacological agent or by genetic modification. Differentiation of the T cells into Th2 cells or function as Th2 cells can be attenuated with a pharmacological agent or by genetic modification. The HLA Class II match can be an HLA-DRB1 allele, an HLA-DQB1 allele, or an HLA-DPB1 allele.

### Activation of Myeloid Cells can be Inhibited in the Pharmaceutical Composition

IL-2, FLT3L, IFN gamma, IFN gamma, IFN alpha, IFN beta, IL-12, TNF alpha, IL-1 beta, IL-6, CSF2, IL-4, IL-5, IL-10, IL-13, IL-21, or TGF-β can be increased in CD4+ T cells by at least about 50% relative to IL-2, IFN gamma, IFN alpha, IFN beta, IL-12, TNF alpha, IL-1 beta, IL-6, FLT3L, IFN gamma, CSF2, IL-4, IL-5, IL-10, IL-13, IL-21, or TGF-β basal activity, respectively. In particular, IL-2 expression can be increased in CD4+ T cells by at least about 50% relative to IL-2 basal activity. The amount of IL-2, IFN gamma, IFN alpha, IFN beta, IL-12, TNF alpha, IL-1 beta, IL-6, FLT3L, IFN gamma, CSF2, IL-4, IL-5, IL-10, IL-13, IL-21 or TGF-β can be measured by Western Blot.

Provided herein are methods for producing a composition comprising: a) obtaining a peripheral blood cell composition from a subject; b) isolating leukocytes from the peripheral blood cell composition, and c) increasing the expression of IL-2, IFN gamma, IFN alpha, IFN beta, IL-12, TNF alpha, IL-1 beta, IL-6, FLT3L, IFN gamma, CSF2, IL-4, IL-5, IL-10, IL-13, IL-21, or TGF-β or combinations thereof. The method can further comprise a treatment to promote differentiation of at least a portion of T cells toward Th1 or Tfh CD4+ T cells, or to maintain CD4+ Th1 or Tfh cells in their state of differentiation. The method can further comprise culturing the leukocytes in vitro. The method can further comprise culturing the leukocytes ex vivo. The method can further comprise adding one or more additional cytokines. The one or more additional cytokines can be IL-2, IL-7, IL-12, IL-15, IL-18, IFNγ, IL-21, or TGF-β. The method can further comprise adding one or more antibodies. The one or more antibodies can be an anti-IL3 antibody, an anti-IL-4 antibody, an anti-CD3 antibody, an anti-CD200 antibody or an anti-CD28 antibody.

The disclosure further provides methods of treating cancer, comprising administering to a subject in need thereof an effective amount of (i) a lymphodepleting agent and (ii) the pharmaceutical composition disclosed herein. The method of treating cancer can comprise administering to a subject in need thereof an effective amount of (i) a lymphodepleting agent, (ii) an inhibitor of NLR family pyrin domain containing 3 (NLRP3), and (iii) the pharmaceutical composition disclosed herein.

The method of treating cancer can comprise administering to a subject in need thereof an effective amount of (i) a lymphodepleting agent, (ii) an agent that inhibits differentiation of the T cells into T regulatory cells or inhibits the function of T regulatory cells, and (iii) the pharmaceutical composition disclosed herein.

The methods can be used to potentiate anti-tumor immunity in a subject having a cancer, comprising administering to a subject in need thereof an effective amount of (i) a lymphodepleting agent, and (ii) the pharmaceutical composition herein.

The lymphodepleting agent can be a cytoreductive agent. The cytoreductive agent can be an alkylating agent, an alkyl sulphonate, a nitrosourea, a triazene, an antimetabolite, a pyrimidine analog, a purine analog, a vinca alkaloids a epipodophyllotoxin, an antibiotics, a dibromomannitol, a deoxyspergualine, a dimethyl myleran or a thiotepa. The alkylating agent can be cyclophosphamide. The purine analog can be fludarabine, cladribine, or pentostatin.

The cancer can be a hematological cancer. The cancer can be a solid cancer. The hematological cancer can be a leukemia, lymphoma, multiple myeloma, myelodysplastic syndrome, or myeloproliferative disorder. The cancer can be leukemia, lymphoma, multiple myeloma, myelodysplastic syndrome or myeloproliferative disorder is non-Hodgkin lymphoma, chronic lymphocytic leukemia, small lymphocytic lymphoma, diffuse large B-cell lymphoma, follicular lymphoma, marginal zone lymphoma, mantle cell lymphoma, acute lymphoblastic leukemia, acute myeloid leukemia, hairy cell leukemia, AIDS-related lymphoma, cutaneous T cell lymphoma, Hodgkin lymphoma, mycosis fungoides, primary central nervous system lymphoma, Sezary syndrome, T cell lymphoma, Waldenström's macroglobulinemia, chronic myeloid leukemia, chronic myelomonocytic leukemia, polycythemia vera, essential thrombocythemia, or idiopathic myelofibrosis.

The solid cancer can be sarcoma, carcinoma, a neurofibromatoma, a colon cancer, a lung cancer, an ovarian cancer, pancreatic cancer, or a breast cancer.

The methods for treating cancer can further comprise administering to the subject in need thereof an additional therapeutic agent. The additional therapeutic agent can be a chemotherapeutic agent, radiation therapy, an immunotherapeutic agent, a T cell agonist cytokine, a CAR-T, a CAR-NK, a bispecific or trispecific T cell or NK cell engager, natural killer cells, gamma-delta T cells, antibody-drug conjugate, an antibody, an immune checkpoint inhibitor, small molecule inhibitor, or an oncolytic virus therapy.

The antibody can be rituximab, Obinutuzumab, ofatumumab, cetuximab, trastuzumab, pertuzumab, brentuximab vedotin, gemtuzumab, trastuzumab emtansine, inotuzumab ozogamicin, glembatumumab vedotin, lorvotuzumab mertansine, cantuzumab mertansine, or milatuzumab-doxorubicin.

The immune checkpoint inhibitor can be an inhibitor of or antibody against PD-L1, PD-1, CTLA-4, LAG-3, TIGIT, or TIM-3.

The small molecule inhibitor can be dasatinib, nilotinib, ponatinib, imatinib, bosutinib, asciminib, lapatinib, or vismodegib.

The pharmaceutical composition disclosed herein can be administered after the lymphodepleting agent.

### 3. DETAILED DESCRIPTION

This disclosure relates to leukocyte compositions that comprise immune cells that are modified for enhanced in vivo anti-tumor activity. In particular, the leukocyte compositions may be enriched for CD4+ Th1 cells and optionally depleted of CD8+ T cells. This disclosure also relates to allogeneic leukocyte compositions and methods for augmenting the efficacy and reducing the toxicity of non-engrafting, CD8-depleted allogeneic leukocyte infusions. This disclosure further relates to allogeneic leukocyte compositions and methods for producing biological molecules such as cytokines by non-engrafting, CD8-depleted allogeneic leukocyte infusions.

Without being bound by theory or mechanism, the inventors believe that the leukocyte compositions can provide a source of CD4+ T cells, or other leukocytes, that can provide signals that influence the immune or other systems in order to achieve a therapeutic outcome, for example, by decreasing immune suppression, and/or increasing immune system activation, and/or reverse the exhaustion of CD8+ T cells in one context. In another context, the therapeutic end may result from influencing the immune system to increase immune suppression and decrease immune system activation. This invention contemplates a variety of different contexts, which may each have unique optimal signatures of biological molecule production upon transfusion in order to achieve a therapeutic outcome (e.g., revive the endogenous anti-tumor response). When the leukocyte composition is part of an allogeneic transplant, the risk of sustained engraftment and graft-versus-host disease is minimized.

The contemplated infusions may achieve greater production of desired biological molecules than would be achieve by simple infusion alone by treating infused cells to overexpress genes associated with those molecules, or to express genes that would not normally be expressed by the infused cells, as the result of treatment of infusion leukocytes, for example by genetic modification using CRISPR, base editing, prime editing, or PASTE, or by treatment with a pharmacological agent.

The leukocyte compositions disclosed herein comprise a plurality of isolated leukocytes which can be obtained from a subject (i.e., autologous), a donor subject (i.e., allogeneic), or another source such as a cord blood or a cell line.

When the leukocytes are obtained from a donor, the leukocytes can be mismatched to a recipient subject for at least one human leukocyte antigen (HLA) Class II allele in the donor versus recipient (graft-versus-host) direction relative to the recipient subject. Alternatively, the donor can comprise at least one HLA class II allele mismatch relative to the recipient in the donor versus the recipient (graft-versus-host) direction and at least one HLA Class II allele match relative to the recipient. The HLA class II allele mismatch or match can be at HLA-DRB1, HLA-DQB1, or HLA-DPB1. For the purposes of this therapy, low expression HLA Class II molecules, for example, HLA-DPA1, HLA-DQA1 and HLA-DRB3, -DRB4, and -DRB5 may not be considered.

In some instances the leukocyte composition disclosed herein can comprise a plurality of isolated leukocytes obtained from a cell line or cord blood and modified, or not modified, to target specific targets. The cell line can be HLA class II allele matched, partially-matched, or mismatched to the subject.

The leukocytes can optionally be depleted of CD8+ T cells by about 10-fold or greater relative to undepleted leukocytes.

The leukocytes can be modified to increase the expression of one or more genes that encode a biological molecule. The one or more genes encoding a biological molecule can, for example, encode a cytokine. Exemplary cytokines include, but are not limited to, L-2, FMS-like tyrosine kinase 3 ligand (FLT3L), interferon (IFN) gamma, colony-stimulating factor 2 (CSF2), IL-4, IL-5, IL-10, IL-13, IFN gamma, IFN alpha, IFN beta, IL-12, TNF alpha, IL-1 beta, IL-6, IL-21, TGF-β, or combinations thereof.

The leukocytes can be modified to increase the expression of one or more cytokines. The leukocytes can be modified to increase the expression of pro-inflammatory cytokines. The leukocytes can be modified to increase the expression of anti-inflammatory cytokines. The leukocytes can be modified to increase the expression of IL-2, FMS-like tyrosine kinase 3 ligand (FLT3L), interferon (IFN) gamma, IFN gamma, IFN alpha, IFN beta, IL-12, TNF alpha, IL-1 beta, IL-6, colony-stimulating factor 2 (CSF2), IL-4, IL-5, IL-10, IL-13, IL-21, TGF-β or combinations thereof. Without wishing to be bound by theory or mechanism, the inventors believe that increasing the expression of IL-2, IFN gamma, IFN alpha, IFN beta, IL-12, TNF alpha, IL-1 beta, IL-6, FLT3L, IFN gamma, CSF2, IL-4, IL-5, IL-10, IL-13, IL-21, TGF-β or combinations thereof may promote naïve CD4+ T cells to differentiate to a state, such as type 1 (Th1) CD4+ T cells, that is favorable for helping effector cells of anti-tumor or anti-viral immunity, or prevent post-naïve CD4+ T cells from converting to cells with suboptimal helper activity for anti-tumor or anti-viral immunity; or help the CD4+ T cells to differentiate to a state, such as type 2 (Th2) that is favorable for a different purpose. For example, a portion of the T cells may be preferentially differentiated to a CD4+ T cell sub-type (e.g. Th1). Increasing the expression of IL-2, IFN gamma, IFN alpha, IFN beta, IL-12, TNF alpha, IL-1 beta, IL-6, FLT3L, IFN gamma, CSF2, IL-21, or combinations thereof may also prevent naïve CD4+ T cells from differentiating to states, such as Th2, Th17, or regulatory T cell, that are suboptimal for promoting anti-tumor or anti-viral immunity, or may prevent CD8+ T cells from becoming exhausted or suppressed by other cells from mediating anti-tumor or anti-viral activity. Increasing the expression of IL-2, FLT3L, IFN gamma, IFN alpha, IFN beta, IL-12, TNF alpha, IL-1 beta, IL-6, IFN gamma, CSF2, IL-4, IL-5, IL-10, IL-13, IL-21 or combinations thereof can optionally provide pro-inflammatory effects to the surrounding microenvironment. IL-2, FLT3L, IFN gamma, IFN alpha, IFN beta, IL-12, TNF alpha, IL-1 beta, IL-6, IFN gamma, CSF2, IL-4, IL-5, IL-10, IL-13, TGF-β or combinations thereof can optionally provide anti-inflammatory effects on the surrounding microenvironment.

The leukocytes can be modified to increase the expression or one or more genes encoding a biological molecule by inserting one or more genes encoding a biological molecule. The one more genes encoding a biological molecule can be the same gene encoding the biological molecule or a different gene encoding a different biological molecule. For example, expression of a cytokine can be increased by inserting one or more genes encoding a biological molecule. The biological molecule can encode the cytokine of interest or can encode a different biological molecule. For illustrative purposes, expression of IL-2 can be increased by inserting a gene encoding IL-2. The expression of IL-2 can be increased by inserting a gene encoding a different biological molecule.

Immune cells that can be modified to increase the expression of one or more genes include, but are not limited to, macrophages, monocytes, granulocytes (e.g., neutrophils, eosinophils, and basophils), and lymphocytes (e.g., T cells and B cells).

Certain illustrative and preferred embodiments are described in further detail herein. The embodiments within the specification should not be construed to limit the scope of the disclosure.

### A. Allogeneic Leukocyte Composition

The disclosure relates to leukocyte compositions.

The leukocytes of the leukocyte composition can be modified to increase the expression of one or more genes that encode a biological molecule. The one or more genes encoding a biological molecule can, for example, encode a cytokine. Exemplary cytokines include, but are not limited to, L-2, FMS-like tyrosine kinase 3 ligand (FLT3L), interferon (IFN) gamma, colony-stimulating factor 2 (CSF2), IFN gamma, IFN alpha, IFN beta, IL-12, TNF alpha, IL-1 beta, IL-6, IL-4, IL-5, IL-10, IL-13, IL-21 or combinations thereof.

In particular, the leukocytes of the leukocyte compositions disclosed herein are modified to increase the expression of IL-2, FLT3L, IFN gamma, IFN gamma, IFN alpha, IFN beta, IL-12, TNF alpha, IL-1 beta, IL-6, CSF2, IL-4, IL-5, IL-10, IL-13, IL-21 or combinations thereof. Without wishing to be bound by theory or mechanism, the inventors believe that increasing the expression of IL-2, FLT3L, IFN gamma, IFN alpha, IFN beta, IL-12, TNF alpha, IL-1 beta, IL-6, IFN gamma, CSF2, IL-4, IL-5, IL-10, IL-13, IL-21 or combinations thereof may promote naïve CD4+ T cells to differentiate to a state, such as type 1 (Th1) CD4+ T cells, that is favorable for helping effector cells of anti-tumor or anti-viral immunity, or prevent post-naïve CD4+ T cells from converting to cells with suboptimal helper activity for anti-tumor or anti-viral immunity. For example, a portion of the T cells may be preferentially differentiated to a CD4+ T cell sub-type, such as Th1. Alternatively, differentiation of T cells into another CD4+ T cell subtype (e.g., Th2 or Treg) can be suppressed. Without wishing to be bound by theory or mechanism the inventors also believe that increasing the expression of IL-2, FLT3L, IFN gamma, CSF2, IL-4, IL-5, IL-10, IL-13, IL-21 or combinations thereof, or combinations thereof in leukocytes (e.g., donor leukocytes) augments their efficacy and may reduce the toxicity of non-engrafting donor leukocyte infusions.

CD4+ T cells are T leukocytes that express T cell receptors recognizing peptide antigens presented in the context of Class II major histocompatibility complex (MHC II) molecules. Tay et. al. (2021), Cancer Gene Therapy, 28:5-17. CD4+ T cells can differentiate into one of several diverse functional subtypes in response to context-dependent signals, which in turn allows them to provide ‘help’ to appropriate effector immune cells in their primary role as central coordinators of the immune response. CD4+ T cells primarily mediate anti-tumor immunity by providing help for CD8+ T cells and antibody responses, by inducing tumoricidal capacity of macrophages, by secretion of effector cytokines such as IFNγ and tumor necrosis factor-α (TNFα), and, under specific contexts, via direct cytotoxicity against tumor cells.

CD4+ T cells can differentiate into Th1 cells that express IFNγ and TNFα, Th2 cells that express IL-4, IL-5, and IL-13; Th9 cells that express IL-9 and IL-21; Th17 cells that expresses IL-17; TFH cells that express IL-6 and IL-21; and Treg cells that express TGFβ and IL-10. Tay et. al. (2021), Cancer Gene Therapy, 28:5-17.

The leukocyte compositions disclosed herein can comprise tumor infiltrating lymphocytes (“TILs”), chimeric receptor T cells (“CAR-T”), or T-cell receptor (“TCR”)-transduced T cells. For example, the leukocyte composition can comprise TILs. For example, the leukocyte composition can comprise CAR-T cells. For example, the leukocyte composition can comprise TCR-transduced T cells.

As disclosed above, the leukocytes of the leukocyte compositions disclosed herein are modified to increase the expression of IL-2, FLT3L, IFN gamma, CSF2, IL-4, IL-5, IL-10, IL-13, IL-21 or combinations thereof.

The leukocyte compositions can be modified to attenuate or increase differentiation of CD4+ T cells into T regulatory (Treg) cells or inhibit CD4+ Treg function.

IL-2, FLT3L, IFN gamma, CSF2, IFN gamma, IFN alpha, IFN beta, IL-12, TNF alpha, IL-1 beta, IL-6, IL-4, IL-5, IL-10, IL-13, IL-21 or combinations thereof can be increased with a pharmacological agent. Alternatively, IL-2, FLT3L, IFN gamma, IFN gamma, IFN alpha, IFN beta, IL-12, TNF alpha, IL-1 beta, IL-6, CSF2, IL-4, IL-5, IL-10, IL-13, IL-21 or combinations thereof can be increased by genetic modification.

CD4+ T cells in the leukocytes can be measured using conventional methodologies known by those skilled in the art, for example, flow cytometry. The expression level of cytokines expressed by the CD4+ T cells can be measured using conventional methodologies by those skilled in the art, for example ELISA or intracellular cytokine staining followed by cell surface staining and flow cytometry.

The leukocyte compositions comprise isolated leukocytes obtained from a subject or a subject donor.

The donor can comprise at least one HLA class II allele mismatch relative to the recipient in the donor versus the recipient (graft-versus-host, or GVH) direction. The HLA class II allele mismatch can be at HLA-DRB1, HLA-DQB1, or HLA-DPB1.

Donors that have not been vaccinated against one or more tumor-specific antigens, including neoantigens, generally have a low frequency of tumor-specific CD4+ T cells. When using a donor whose immune system has not been vaccinated against one or more tumor-specific antigens, no HLA Class II matching is required between the donor and the recipient because the ability to revive endogenous anti-tumor immunity is based on the activity of alloreactive CD4+ T cells.

Some degree of HLA Class II allele matching is required when vaccinating the donor against tumor-specific antigens or when expanding tumor-specific CD4+ T cells ex vivo since the expanded tumor-specific CD4+ T cells are restricted to donor HLA Class II molecules and are predicted to be ineffective at delivering help in the recipient unless the recipient expresses at least one HLA Class II molecule that is shared by the donor. Among HLA Class II molecules, the preferred molecules for sharing are the high expression molecules HLA-DRB1>HLA-DPB1>HLA-DQB1>>>>HLADRB3,4,5=HLA-DQA1.

The donor can be partially or completely mismatched at HLA class II alleles in the donor anti-recipient (GVH) direction, for example HLA-DRB1, HLA-DQB1, and HLA-DPB1. The donor can be partially or completely mismatched at HLA class II alleles, for example HLA-DRB1, HLA-DQB1, and HLA-DPB1 and completely matched for Class I alleles. The donor can be completely mismatched with unshared HLAs of first-degree relatives of the recipient who are potential donors for allogeneic stem cell transplantation.

The donor leukocytes may be stimulated in vivo or ex vivo to increase the frequency, compared to the unstimulated leukocytes, of CD4+ T cells that proliferate and/or secrete IFNγ in response to a tumor or viral antigen. The stimulation may consist of deliberate in vivo vaccination of the donor against a tumor antigen or a viral antigen. Alternatively, or in addition, donor leukocytes containing CD4+ T cells can be stimulated ex vivo using antigen-presenting cells (APCs), such as dendritic cells, pulsed with a tumor or viral antigen in the presence or absence of CD4+ T cell-polarizing cytokines. The tumor antigen or viral antigen can be present in the recipient. In instances in which the donor is immunized or donor cells are stimulated ex vivo with antigen pulsed-APCs, the donor must comprise at least one HLA Class II allele match relative to the recipient. The HLA class II allele match can be at HLA-DRB1, HLA-DQB1, or HLA-DPB1. In embodiments, the immunized donor can have at least one HLA class II allele mismatch relative to the recipient in the donor versus the recipient (graft-versus-host) direction and at least one HLA Class II allele match relative to the recipient. When the donor leukocytes have not been stimulated to increase the frequency of tumor- or virus-specific CD4+ T cells, the donor HLA Class II molecules HLA-DRB1, HLA-DQB1, and HLA-DPB1 may be fully mismatched to the recipient in the donor anti-recipient (GVH) direction.

In embodiments, the leukocyte composition can comprise leukocytes obtained from a cell line or from cord blood.

A donor sample can be obtained from a cord blood bank. When the donor sample is obtained from a cord blood bank, a desirable sample may include non-frequent and/or rare HLA alleles as a subject is less likely to contain serum antibodies to non-frequent and/or rare HLA allele types. Exemplary rare alleles include, but are not limited to, A*24:41, B*07:02:28, B*35:03:03, B*39: 40N, DRB1*13:23, DRB1*14:111, B*44:16 and DRB1*01:31, C*06:49N, B*37:03N, A*24:312N, and A*30:76N.

In embodiments, the recipient may not have detectable antibodies reactive against HLA of the donor. Detectable antibodies can be determined using conventional methods known to those of skill in the art. For example, the recipient may not have antibodies against donor HLA molecules that are detectable by complement-dependent cytotoxicity, in flow cytometric cross-match assays as a positive result is undesirable, or mean fluorescence intensity (MFI) of 3000 or greater in a solid phase immunoassay is unacceptable.

The number of natural killer cells in the composition can be less than or equal to the number of natural killer cells in the peripheral blood composition.

In embodiments, the CD4+ T cells present in the compositions are not activated ex vivo.

The leukocytes present in the leukocyte composition are depleted of CD8+ T cells. CD8+ T cells can be depleted using any known methods. For example, magnetic bead cell sorters or flow cytometry may be used to deplete the CD8+ T cells. Reducing CD8+ T cells can involve using an anti-CD8+ antibody associated with a magnetic particle or an anti-CD8+ antibody plus complement.

The leukocytes can be depleted of CD8+ T cells by about 1 fold, about 2 fold, about 3 fold, about 4 fold, about 5 fold, about 6 fold, about 7 fold, about 8 fold, about 9 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 200 fold, about 300 fold, about 400 fold, about 500 fold, about 600 fold, about 700 fold, about 800 fold, about 900 fold, about 1,000 fold or greater relative to undepleted leukocytes.

### B. Therapeutic Applications

The disclosure also relates to methods for treating a disease or condition, such as cancer, and/or infectious diseases, and/or autoimmune diseases.

The method comprises administering to a subject in need thereof a lymphodepleting agent and/or an immune-stimulating agent and administering to the subject an leukocyte composition as described herein.

The lymphodepleting agent can be a cytoreductive agent. Exemplary cytoreductive agents include, but are not limited to, an alkylating agent, alkyl sulphonates, nitrosoureas, triazene, antimetabolites, pyrimidine analog, purine analog, vinca alkaloids, epiodophyllotoxins, antibiotics, dirbromannitol, deoxyspergualine, dimethyl myleran and tiotepa.

The lymphodepleting agent can be a chemotherapeutic agent or a biologic agent. Exemplary chemotherapeutic agents and/or biologic agents include, but are not limited to, an antibody, a B cell receptor pathway inhibitor, a T cell receptor inhibitor, a PI3K inhibitor, an IAP inhibitor, an mTOR inhibitor, a radioimmunotherapeutic, a DNA damaging agent, a histone deacetylase inhibitor, a protein kinase inhibitor, a hedgehog inhibitor, an Hsp90 inhibitor, a telomerase inhibitor, a Jakl/2 inhibitor, a protease inhibitor, an IRAK inhibitor, a PKC inhibitor, a PARP inhibitor, a CYP3 A4 inhibitor, an AKT inhibitor, an Erk inhibitor, a proteosome inhibitor, an alkylating agent, an anti-metabolite, a plant alkaloid, a terpenoid, a cytotoxin, a topoisomerase inhibitor, a CD79A inhibitor, a CD79B inhibitor, a CD 19 inhibitor, a Lyn inhibitor, a Syk inhibitor, a PI3K inhibitor, a Blnk inhibitor, a PLCy inhibitor, a PKCP inhibitor, a CD22 inhibitor, a Bcl-2 inhibitor, an IRAK 1/4 inhibitor, a JAK inhibitor (e.g., ruxolitinib, baricitinib, CYT387, lestauritinib, pacritinib, TG101348, SAR302503, tofacitinib (Xeljanz), etanercept (Enbrel), GLPG0634, R256), a microtubule inhibitor, a Topo II inhibitor, anti-TWEAK antibody, anti-IL 17 bispecific antibody, a CK2 inhibitor, anaplastic lymphoma kinase (ALK) and c-Met inhibitors, demethylase enzyme inhibitors such as demethylase, HDM, LSDI and KDM, fatty acid synthase inhibitors such as spirocyclic piperidine derivatives, glucocorticosteriod receptor agonist, fusion anti-CD 19-cytotoxic agent conjugate, antimetabolite, p70S6K inhibitor, immune modulators, AKT/PKB inhibitor, procaspase-3 activator PAC-1, BRAF inhibitor, lactate dehydrogenase A (LDH-A) inhibitor, CCR2 inhibitor, CXCR4 inhibitor, chemokine receptor antagonists, DNA double stranded break repair inhibitors, NOR202, GA-101, TLR2 inhibitor, Muromonab-CD3, rituximab (rituxan), carfilzomib, fludarabine, cyclophosphamide, vincristine, chlorambucil, ifosphamide, doxorubicin, mesalazine, thalidomide, revlimid, lenalidomide, temsirolimus, everolimus, fostamatinib, paclitaxel, docetaxel, ofatumumab, dexamethasone, bendamustine, CAL-101, ibritumomab, tositumomab, bortezomib, pentostatin, endostatin, ritonavir, ketoconazole, an anti-VEGF antibody, herceptin, cetuximab, cisplatin, carboplatin, docetaxel, erlotinib, etopiside, 5-fluorouracil, gemcitabine, ifosphamide, imatinib mesylate (Gleevec), gefitinib, erlotinib, procarbazine, irinotecan, leucovorin, mechlorethamine, methotrexate, oxaliplatin, paclitaxel, sorafenib, sunitinib, topotecan, vinblastine, GA-1101, dasatinib, Sipuleucel-T, disulfiram, epigallocatechin-3-gallate, salinosporamide A, ONX0912, CEP-18770, MLN9708, R-406, lenalinomide, spirocyclic piperidine derivatives, quinazoline carboxamide azetidine compounds, thiotepa, DWA2114R, NK121, IS 3 295, 254-S, alkyl sulfonates such as busulfan, improsulfan and piposulfan, aziridines such as benzodepa, carboquone, meturedepa and uredepa, ethylenimine, methylmelamines such as altretamine, triethylenemelamine, triethylenephosphoramide, triethylenethiophosphoramide and trimethylmelamine, chlornaphazine, estramustine, ifosfamide, mechlorethamine, oxide hydrochloride, novobiocin, phenesterine, prednimustine, trofosfamide, uracil mustard, nitrosoureas such as carmustine, chlorozotocin, fotemustine, lomustine, nimustine, ranimustine, antibiotics such as aclacinomycins, actinomycin, anthramycin, azaserine, bleomycins, cactinomycin, calicheamicin, carubicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin, antimetabolites such as methotrexate and 5-fluorouracil (5-FU), folic acid analogues such as denopterin, methotrexate, pteropterin, trimetrexate, purine analogs such as fludarabine, 6-mercaptopurine, thiamiprine, thioguanine, pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone, anti-adrenals such as aminoglutethimide, mitotane, trilostane, folic acid replenisher such as folinic acid, aceglatone, aldophosphamide glycoside, aminolevulinic acid, amsacrine, bestrabucil, bisantrene, edatrexate, defosfamide, demecolcine, diaziquone, eflornithine, elliptinium acetate, etoglucid, gallium nitrate, hydroxyurea, lentinan, lonidamine, mitoguazone, mitoxantrone, mopidamol, nitracrine, pentostatin, phenamet, pirarubicin, podophyllinic acid, 2-ethylhydrazide, procarbazine, polysaccharide-K, razoxane, sizofiran, spirogermanium, tenuazonic acid, triaziquone, 2,2′,2″-trichlorotriethylamine, urethan, vindesine, dacarbazine, mannomustine, mitobronitol, mitolactol, pipobroman, gacytosine, cytosine arabinoside, taxoids, e.g., paclitaxel and docetaxel, 6-thioguanine, mercaptopurine, methotrexate, platinum analogs, platinum, etoposide (VP-16), ifosfamide, mitomycin C, mitoxantrone, vincristine, vinorelbine, Navelbine, Novantrone, teniposide, daunomycin, aminopterin, Xeloda, ibandronate, CPT1 1, topoisomerase inhibitor RFS 2000, difluoromethylornithine (DMFO), retinoic acid, esperamycins, capecitabine, and pharmaceutically acceptable salts, acids or derivatives of, anti-hormonal agents such as anti-estrogens including for example tamoxifen, raloxifene, aromatase inhibiting 4(5)-imidazoles, 4-hydroxytamoxifen, trioxifene, keoxifene, LY117018, onapristone and toremifene (Fareston), antiandrogens such as flutamide, nilutamide, bicalutamide, leuprolide and goserelin, ACK inhibitors such as AVL-263 (Avila Therapeutics/Celgene Corporation), AVL-292 (Avila Therapeutics/Celgene Corporation), AVL-291 (Avila Therapeutics/Celgene Corporation), BMS-488516 (Bristol-Myers Squibb), BMS-509744 (Bristol-Myers Squibb), CGI-1746 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited) or a combination thereof.

The compositions and methods disclosed herein can used for any suitable cancer, including, but not limited to, bladder cancer, brain cancer, breast cancer, colorectal cancer, cervical cancer, gastrointestinal cancer, genitourinary cancer, head and neck cancer, lung cancer, ovarian cancer, prostate cancer, renal cancer, skin cancer, and testicular cancer, cardiac cancers, including, for example sarcoma, e.g., angiosarcoma, fibrosarcoma, rhabdomyosarcoma, and liposarcoma, myxoma, rhabdomyoma, fibroma, lipoma and teratoma, lung cancers, including, for example, bronchogenic carcinoma, e.g., squamous cell, undifferentiated small cell, undifferentiated large cell, and adenocarcinoma, alveolar and bronchiolar carcinoma, bronchial adenoma, sarcoma, lymphoma, chondromatous hamartoma, and mesothelioma, gastrointestinal cancer, including, for example, cancers of the esophagus, e.g., squamous cell carcinoma, adenocarcinoma, leiomyosarcoma, and lymphoma, cancers of the stomach, e.g., carcinoma, lymphoma, and leiomyosarcoma, cancers of the pancreas, e.g., ductal adenocarcinoma, insulinoma, glucagonoma, gastrinoma, carcinoid tumors, and vipoma, cancers of the small bowel, e.g., adenocarcinoma, lymphoma, carcinoid tumors, Kaposi's sarcoma, leiomyoma, hemangioma, lipoma, neurofibroma, and fibroma, cancers of the large bowel, e.g., adenocarcinoma, tubular adenoma, villous adenoma, hamartoma, and leiomyoma, genitourinary tract cancers, including, for example, cancers of the kidney, e.g., adenocarcinoma, Wilm's tumor (nephroblastoma), lymphoma, and leukemia, cancers of the bladder and urethra, e.g., squamous cell carcinoma, transitional cell carcinoma, and adenocarcinoma, cancers of the prostate, e.g., adenocarcinoma, and sarcoma, cancer of the testis, e.g., seminoma, teratoma, embryonal carcinoma, teratocarcinoma, choriocarcinoma, sarcoma, interstitial cell carcinoma, fibroma, fibroadenoma, adenomatoid tumors, and limphoma, liver cancers, including, for example, hepatoma, e.g., hepatocellular carcinoma, cholangiocarcinoma, hepatoblastoma, angiosarcoma, hepatocellular adenoma, and hemangioma, bone cancers, including, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors, nervous system cancers, including, for example, cancers of the skull, e.g., osteoma, hemangioma, granuloma, xanthoma, and osteitis defoinians, cancers of the meninges, e.g., meningioma, meningiosarcoma, and gliomatosis, cancers of the brain, e.g., astrocytoma, medulloblastoma, glioma, ependymoma, germinoma (pinealoma), glioblastoma multiform, oligodendroglioma, schwannoma, retinoblastoma, and congenital tumors, and cancers of the spinal cord, e.g., neurofibroma, meningioma, glioma, and sarcoma, gynecological cancers, including, for example, cancers of the uterus, e.g., endometrial carcinoma, cancers of the cervix, e.g., cervical carcinoma, and pre tumor cervical dysplasia, cancers of the ovaries, e.g., ovarian carcinoma, including serous cystadenocarcinoma, mucinous cystadenocarcinoma, unclassified carcinoma, granulosa thecal cell tumors, Sertoli Leydig cell tumors, dysgerminoma, and malignant teratoma, cancers of the vulva, e.g., squamous cell carcinoma, intraepithelial carcinoma, adenocarcinoma, fibrosarcoma, and melanoma, cancers of the vagina, e.g., clear cell carcinoma, squamous cell carcinoma, botryoid sarcoma, and embryonal rhabdomyosarcoma, and cancers of the fallopian tubes, e.g., carcinoma, hematologic cancers, including, for example, cancers of the blood, e.g., acute myeloid leukemia, chronic myeloid leukemia, acute lymphoblastic leukemia, chronic lymphocytic leukemia, myeloproliferative diseases, multiple myeloma, and myelodysplasia syndrome, Hodgkin's lymphoma, non-Hodgkin's lymphoma (malignant lymphoma) and Waldenstrom's macro globulinemia, skin cancers, including, for example, malignant melanoma, basal cell carcinoma, squamous cell carcinoma, Kaposi's sarcoma, moles dysplastic nevi, lipoma, angioma, dermatofibroma, keloids, psoriasis, and adrenal gland cancers, including, for example, neuroblastoma. In certain embodiments, when the disease is cancer, it may include a lung cancer tumor, a breast cancer tumor, a prostate cancer tumor, a brain cancer tumor, or a skin cancer tumor for example.

The subject can have a solid tumor. In some embodiments, the subject can have a sarcoma, carcinoma, or a neurofibromatoma. In some embodiments, the subject can have a colon cancer. In some embodiments, the subject can have a lung cancer. In some embodiments, the subject can have an ovarian cancer. In some embodiments, the subject can have a pancreatic cancer. In some embodiments, the subject can have a prostate cancer. In some embodiments, the subject can have a proximal or distal bile duct carcinoma. In some embodiments, the subject can have a breast cancer. In some embodiments, the subject can have a HER2-positive breast cancer. In some embodiments, the subject can have a HER2-negative breast cancer. In some embodiments, the subject has been treated for a solid tumor, and the method is applied to treat a subject as adjuvant therapy, that is the method is applied to the subject when the cancer is in a complete remission so as to prevent relapse of the cancer.

The subject can have a hematologic cancer. In some embodiments, the cancer is a leukemia, a lymphoma, a myeloma, a myelodysplastic syndrome, or a myeloproliferative neoplasm. In some embodiments, the cancer is a non-Hodgkin lymphoma. In some embodiments, the cancer is a Hodgkin lymphoma. In some embodiments, the cancer is a B-cell malignancy. In some embodiments, the B-cell malignancy is chronic lymphocytic leukemia (CLL), small lymphocytic lymphoma (SLL), diffuse large B-cell lymphoma (DLBCL), follicular lymphoma (FL), activated B-cell diffuse large B-cell lymphoma (ABC-DLBCL), germinal center diffuse large B-cell lymphoma (GCB DLBCL), primary mediastinal B-cell lymphoma (PMBL), Burkitt's lymphoma, immunoblastic large cell lymphoma, precursor B-lymphoblastic lymphoma, mantle cell lymphoma (MCL), B cell prolymphocytic leukemia, lymphoplasmacytic lymphoma, Waldenstrom macroglobulinemia, splenic marginal zone lymphoma, plasma cell myeloma, plasmacytoma, extranodal marginal zone B cell lymphoma, nodal marginal zone B cell lymphoma, mediastinal (thymic) large B cell lymphoma, intravascular large B cell lymphoma, primary effusion lymphoma, or lymphomatoid granulomatosis. In some embodiments, the cancer is a T cell malignancy. In some embodiments, the T cell malignancy is peripheral T cell lymphoma not otherwise specified (PTCL-NOS), anaplastic large cell lymphoma, angioimmunoblastic lymphoma, cutaneous T cell lymphoma, adult T cell leukemia/lymphoma (ATLL), blastic NK-cell lymphoma, enteropathy-type T cell lymphoma, hematosplenic gamma-delta T cell lymphoma, lymphoblastic lymphoma, nasal NK/T cell lymphomas, or treatment-related T cell lymphomas. In some embodiments, the subject can have multiple myeloma.

The subject can have a relapsed or refractory cancer.

The methods disclosed herein can further involve the administration of one or more additional agents to treat cancer, such as chemotherapeutic agents (e.g., Adriamycin, Cerubidine, Bleomycin, Alkeran, Velban, Oncovin, Fluorouracil, Thiotepa, Methotrexate, Bisantrene, Noantrone, Thiguanine, Cytaribine, Procarabizine), immuno-oncology agents (e.g., anti-PD-L1, anti-CTLA4, anti-PD-1, anti-CD47, anti-GD2), cellular therapies (e.g., CAR-T, T cell therapy, natural killer cell therapy, gamma delta T cell therapy), oncolytic viruses and the like.

Non-limiting examples of additional agents to treat cancer include acivicin, aclarubicin, acodazole hydrochloride, acronine, adozelesin, aldesleukin, altretamine, ambomycin, ametantrone acetate, aminoglutethimide, amsacrine, anastrozole, anthramycin, asparaginase, asperlin, azacitidine, azetepa, azotomycin, batimastat, benzodepa, bicalutamide, bisantrene hydrochloride, bisnafide dimesylate, bizelesin, bleomycin sulfate, brequinar sodium, bropirimine, busulfan, cactinomycin, calusterone, caracemide, carbetimer, carboplatin, carmustine, carubicin hydrochloride, carzelesin, cedefingol, chlorambucil, cirolemycin, cisplatin, cladribine, crisnatol mesylate, cyclophosphamide, cytarabine, dacarbazine, dactinomycin, daunorubicin hydrochloride, decitabine, dexormaplatin, dezaguanine, dezaguanine mesylate, diaziquone, docetaxel, doxorubicin, doxorubicin hydrochloride, droloxifene, droloxifene citrate, dromostanolone propionate, duazomycin, edatrexate, eflornithine hydrochloride, elsamitrucin, enloplatin, enpromate, epipropidine, epirubicin hydrochloride, erbulozole, esorubicin hydrochloride, estramustine, estramustine phosphate sodium, etanidazole, etoposide, etoposide phosphate, etoprine, fadrozole hydrochloride, fazarabine, fenretinide, floxuridine, fludarabine phosphate, fluorouracil, flurocitabine, fosquidone, fostriecin sodium, gemcitabine, gemcitabine hydrochloride, hydroxyurea, idarubicin hydrochloride, ifosfamide, ilmofosine, interleukin II (including recombinant interleukin II, or rIL2), interferon alpha-2a, interferon alpha-2b, interferon alpha-n1 interferon alpha-n3, interferon beta-Ia, interferon gamma-Ib, iproplatin, irinotecan hydrochloride, lanreotide acetate, letrozole, leuprolide acetate, liarozole hydrochloride, lometrexol sodium, lomustine, losoxantrone hydrochloride, masoprocol, maytansine, mechlorethamine hydrochloride, megestrol acetate, melengestrol acetate, melphalan, menogaril, mercaptopurine, methotrexate, methotrexate sodium, metoprine, meturedepa, mitindomide, mitocarcin, mitocromin, mitogillin, mitomalcin, mitomycin, mitosper, mitotane, mitoxantrone hydrochloride, mycophenolic acid, nocodazole, nogalamycin, ormaplatin, oxisuran, paclitaxel, pegaspargase, peliomycin, pentamustine, peplomycin sulfate, perfosfamide, pipobroman, piposulfan, piroxantrone hydrochloride, plicamycin, plomestane, porfimer sodium, porfiromycin, prednimustine, procarbazine hydrochloride, puromycin, puromycin hydrochloride, pyrazofurin, riboprine, rogletimide, safingol, safingol hydrochloride, semustine, simtrazene, sparfosate sodium, sparsomycin, spirogermanium hydrochloride, spiromustine, spiroplatin, streptonigrin, streptozocin, sulofenur, talisomycin, tecogalan sodium, tegafur, teloxantrone hydrochloride, temoporfin, teniposide, teroxirone, testolactone, thiamiprine, thioguanine, thiotepa, tiazofurin, tirapazamine, toremifene citrate, trestolone acetate, triciribine phosphate, trimetrexate, trimetrexate glucuronate, triptorelin, tubulozole hydrochloride, uracil mustard, uredepa, vapreotide, verteporfin, vinblastine sulfate, vincristine sulfate, vindesine, vindesine sulfate, vinepidine sulfate, vinglycinate sulfate, vinleurosine sulfate, vinorelbine tartrate, vinzolidine sulfate, vinzolidine sulfate, vorozole, zeniplatin, zinostatin, zorubicin hydrochloride.

The methods disclosed herein can further comprise administration of an anti-tumor antibody/drug conjugate. The anti-tumor antibody/drug conjugate can include, but is not limited to, rituximab, cetuximab, trastuzumab, and pertuzumab, brentuximab vedotin, gemtuzumab ozogamicin, trastuzumab emtansine, inotuzumab ozogamicin, glembatumumab vedotin, lorvotuzumab mertansine, cantuzumab mertansine, or milatuzumab-doxorubicin.

The methods disclosed herein can further comprise administering an antiviral agent. Exemplary anti-viral agents include, but are not limited to, acyclovir, famciclovir, ganciclovir, penciclovir, valacyclovir, valganciclovir, idoxuridine, trifluridine, brivudine, cidofovir, docosanol, fomivirsen, foscarnet, tromantadine, imiquimod, podophyllotoxin, entecavir, lamivudine, telbivudine, clevudine, adefovir, tenofovir, boceprevir, telaprevir, pleconaril, arbidol, amantadine, rimantadine, oseltamivir, zanamivir, peramivir, inosine, interferon (e.g., Interferon alfa-2b, Peginterferon alfa-2a), ribavirin/taribavirin, abacavir, emtricitabine, lamivudine, didanosine, zidovudine, apricitabine, stampidine, elvucitabine, racivir, amdoxovir, stavudine, zalcitabine, tenofovir, efavirenz, nevirapine, etravirine, rilpivirine, loviride, delavirdine, atazanavir, fosamprenavir, lopinavir, darunavir, nelfmavir, ritonavir, saquinavir, tipranavir, amprenavir, indinavir, enfuvirtide, maraviroc, vicriviroc, PRO 140, ibalizumab, raltegravir, elvitegravir, bevirimat, and vivecon.

The methods disclosed herein can be suitable for treating an autoimmune disease (e.g., graft versus host disease, multiple sclerosis, rheumatoid arthritis, myasthenia gravis, Crohn's disease, lupus) or can be at risk of graft rejection.

The compositions disclosed herein are typically administered systemically, for example by intravenous injection or intravenous infusion. Other routes of administration can be used, such as orally, parenterally, intravenous, intravenously, intra-articularly, intraperitoneally, intramuscularly, subcutaneously, intracavity, transdermally, intrahepatically, intracranially, nebulization/inhalation, by installation via bronchoscopy, or intratumorally.

The dosage regimen will be determined by the attending physician and other clinical factors. Dosages for any one patient depends on many factors, including the patient's size, body surface area, age, sex, the particular compound to be administered, time and route of administration, the kind of therapy, general health and other drugs being administered concurrently. An “effective dose” refers to amounts of the active ingredient that are sufficient to affect the course and the severity of the disease, leading to the reduction or remission of such pathology and may be determined using known methods.

### C. Method of Preparing

The disclosure also relates to methods of preparing the leukocyte compositions disclosed herein. The method comprises obtaining a peripheral blood cell composition from a subject or from a donor subject. When the peripheral blood cell composition is from a donor subject, the donor subject is generally allogeneic to a recipient subject or from a cell line or umbilical cord blood.

The peripheral blood cell composition can be a whole blood product or an apheresis product. The peripheral blood cell composition can be obtained using means known in the art, for example through venipuncture. The peripheral blood cell composition comprises both CD8+ T cells and CD4+ T cells. The peripheral blood cell composition can be obtained from human or non-human subjects. Preferentially, the peripheral blood cell composition is obtained from a human.

The leukocytes from the donor subject can be mismatched to a recipient subject for at least one HLA Class II allele mismatch in the donor versus recipient (graft-versus-host) direction relative to the recipient subject. Alternatively, the donor can have at least one HLA class II allele mismatch relative to the recipient in the donor versus the recipient (graft-versus-host) direction and at least one HLA Class II allele match relative to the recipient. The HLA class II allele mismatch or match can be at HLA-DRB1, HLA-DQB1, or HLA-DPB1.

If the donor and recipient are ABO blood type incompatible and the leukocyte composition comprises a number of red blood cells, then making the leukocyte composition can further comprise reducing the number of red blood cells. “ABO blood type incompatible” as used herein refers to when the recipient has a major ABO red blood cell incompatibility against the donor, e.g., the recipient is blood type O, and the donor is blood type A, B, or AB; the recipient is type A and the donor is type B or AB; or the recipient is type B and the donor is type A or AB. The number of red blood cells can comprise less than or equal to about 50 ml in packed volume. e.g., less than or equal to about 50 ml in packed volume, preferably less than or equal to about 30 ml in packed volume, further “packed volume” should be defined, for example, centrifugation of the leukocyte composition would result in a packed volume of 50 ml or less of red blood cells, a measured volume sample of the leukocyte composition could also be screened to provide a proportionally representative volume of packed blood cells.

Mononuclear cells are then isolated from the peripheral blood cell composition, for example by Ficoll-Hypaque gradient separation. Next, the number of CD8+ cells in the leukocytes can optionally be depleted. The number of CD8+ cells in the leukocytes can be depleted by about 1 fold, about 2 fold, about 3 fold, about 4 fold, about 5 fold, about 6 fold, about 7 fold, about 8 fold, about 9 fold, about 10 fold, about 15 fold, about 20 fold, about 25 fold, about 30 fold, about 35 fold, about 40 fold, about 45 fold, about 50 fold, about 55 fold, about 60 fold, about 65 fold, about 70 fold, about 75 fold, about 80 fold, about 85 fold, about 90 fold, about 95 fold, about 100 fold, about 200 fold, about 300 fold, about 400 fold, about 500 fold, about 600 fold, about 700 fold, about 800 fold, about 900 fold, about 1,000 fold or greater relative to undepleted leukocytes.

The leukocytes are further modified to increase the expression of IL-2, FLT3L, IFN gamma, CSF2, IL-4, IL-5, IL-10, IL-13, IL-21, IFN gamma, IFN alpha, IFN beta, IL-12, TNF alpha, IL-1 beta, IL-6, TGF-β, or combinations thereof. Without wishing to be bound by theory or mechanism, the inventors believe that increasing the expression of IL-2, FLT3L, IFN gamma, CSF2, IL-4, IL-5, IL-10, IL-13, IL-21, IFN gamma, IFN alpha, IFN beta, IL-12, TNF alpha, IL-1 beta, IL-6, TGF-β, or combinations thereof may promote naïve CD4+ T cells to differentiate to a state, such as type 1 (Th1) CD4+ T cells, that is favorable for helping effector cells of anti-tumor or anti-viral immunity, or prevent post-naïve CD4+ T cells from converting to cells with suboptimal helper activity for anti-tumor or anti-viral immunity. For example, a portion of the T cells may be preferentially differentiated to a CD4+ T cell sub-type, specifically Th1.

The method can comprise promoting differentiation of at least a portion of T cells toward Th1 CD4+ T cells. Increasing the expression of IL-2, FLT3L, IFN gamma, IFN alpha, IFN beta, IL-12, TNF alpha, IL-1 beta, IL-6, IFN gamma, CSF2, IL-4, IL-5, IL-10, IL-13, IL-21, TGF-β, or combinations thereof can promote differentiation of a portion of T cells towards Th1 CD4+ T cells.

The method can comprise culturing the leukocytes in vitro.

The method of producing the leukocyte composition can further comprise stimulating antigen-specific leukocytes in the composition with antigen-presenting cells pulsed with antigenic peptides.

The method of producing the leukocyte composition can further comprise adding one or more additional agents, such as a cytokine or antibodies.

The additional agent can be a cytokine. Exemplary cytokines that can be added include IL-2, IFN gamma, IFN alpha, IFN beta, IL-12, TNF alpha, IL-1 beta, IL-6, IL-7, IL-12, IL-15, IL-18, IFNγ, IL-21, CCDC134, GM-CSF, or LYG1.

The additional agent can be an antibody. Exemplary antibodies include an anti-IL3 antibody, an anti-IL-4 antibody, an anti-CD3 antibody, an anti-CD200 antibody or an anti-CD28 antibody.

The additional agent can be an inhibitor. Exemplary inhibitors include inhibitors of MEK 1/2, ERK, p38, Cox-2, Pi13k, c512, setdb1, or Got1.

Other exemplary agents include, but are not limited to, receptor agonists (e.g., RAR alpha or TLR), transcription factors (e.g., T-bet and Tbx21), lipoarabinomannans, or lipomannans derived from BCG cell bodies

### D. Definitions

Various terms relating to aspects of the description are used throughout the specification and claims. Such terms are to be given their ordinary meaning in the art unless otherwise indicated. Other specifically defined terms are to be construed in a manner consistent with the definitions provided herein. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a,” “an,” and “the” include plural forms unless the context clearly indicates otherwise. The terms “include,” “such as,” and the like are intended to convey inclusion without limitation, unless otherwise specifically indicated.

Unless otherwise indicated, the terms “at least,” “less than,” and “about,” or similar terms preceding a series of elements or a range are to be understood to refer to every element in the series or range. Those skilled in the art will recognize, or be able to ascertain using no more than routine experimentation, many equivalents to the specific embodiments of the invention described herein. Such equivalents are intended to be encompassed by the following claims.

The term “cancer” refers to the physiological condition in mammals in which a population of cells is characterized by uncontrolled proliferation, immortality, metastatic potential, rapid growth and proliferation rate and/or certain morphological features. Often cancers can be in the form of a tumor or mass, but may exist alone within the subject, or may circulate in the blood stream as independent cells, such as leukemic or lymphoma cells. The term cancer includes all types of cancers and metastases, including hematological malignancy, solid tumors, sarcomas, carcinomas and other solid and non-solid tumors. Examples of cancers include, but are not limited to, carcinoma, lymphoma, blastoma, sarcoma, and leukemia. More particular examples of such cancers include squamous cell cancer, small cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer (e.g., triple negative breast cancer), osteosarcoma, melanoma, colon cancer, colorectal cancer, endometrial (e.g., serous) or uterine cancer, salivary gland carcinoma, kidney cancer, liver cancer, prostate cancer, vulval cancer, thyroid cancer, hepatic carcinoma, and various types of head and neck cancers. Triple negative breast cancer refers to breast cancer that is negative for expression of the genes for estrogen receptor (ER), progesterone receptor (PR), and Her2/neu.

As used herein, the term “T cell exhaustion” refers to the progressive loss of effector function (loss of IL-2, TNF-α, and IFN-γ production, or failure to kill cells expressing the T cell's cognate antigen) and sustained expression of inhibitory receptors such as PD-1, T cell immunoglobulin domain, and mucin domain-containing protein 3 (Tim-3), CTLA-4, lymphocyte-activation gene 3 (LAG-3), and CD160 with a transcriptional program distinct from functional effector or memory T cells

The term “subject” herein to refers to any animal, such as any mammal, including but not limited to, humans, non-human primates, rodents, and the like. In some embodiments, the mammal is a mouse. In some embodiments, the mammal is a human.

As used herein, the term “therapeutically effective amount” refers to an amount of a compound described herein (i.e., a leukocyte composition) that is sufficient to achieve a desired pharmacological or physiological effect under the conditions of administration. For example, a “therapeutically effective amount” can be an amount that is sufficient to reduce the signs or symptoms of a disease or condition (e.g., a tumor). Those skilled in the art will appreciate that the therapeutic effects need not be complete or curative, as long as some benefit is provided to the subject. A therapeutically effective amount of a pharmaceutical composition can vary according to factors such as the disease state, age, sex, and weight of the individual, and the ability of the pharmaceutical composition to elicit a desired response in the individual. An ordinarily skilled clinician can determine appropriate amounts to administer to achieve the desired therapeutic benefit based on these and other considerations.

### 4. EQUIVALENTS

It will be readily apparent to those skilled in the art that other suitable modifications and adaptions of the methods of the invention described herein are obvious and may be made using suitable equivalents without departing from the scope of the disclosure or the embodiments. Having now described certain compounds and methods in detail, the same will be more clearly understood by reference to the following examples, which are introduced for illustration only and not intended to be limiting.

### 5. EXAMPLES

### Example 1. Validating Allogeneic Adoptive T Leukocytes in Localized Production and Delivery of Inflammatory Cytokines

The objective of this experiment is to validate the treatment benefit resulting from infusion of an adoptive cell therapy composition composed of alloreactive CD4+ T cells engineered to express an inflammatory cytokine, IL-12.

This experiment uses the TC-1-Luc lung cancer cell line infused into C57BL/6 mice as a model for human tumors, including lung cancer. Allogeneic BALB/c CD4+ T cells are used as a platform for production and delivery of an inflammatory cytokine not normally produced by CD4+ T cells, IL-12.

Allogeneic CD4+ T cells expressing IL-12 will be generated by knocking a recombinant single-chain IL-12 sequence into the PDCDI locus of BALB/c primary CD4+ T cells (IL-12-BALBs) as described previously, using high-efficiency non-viral CRISPR/Cas9-mediated gene editing, as described elsewhere. Kim et al., (2023), Front Immunol., 14:1062365; Oh et al., (2022), J. Exp. Med., 219(5):e20211530.

CD4+ T cells from donors will be isolated by negative selection using Miltenyi CD4+ T Cell Isolation Kits, mouse (Miltenyi, cat. no. 130-104-454) as per Miltenyi protocol. T cells will be activated 1 day post-nucleofection with Miltenyi T Cell Activation/Expansion kits (cat. no. 130-093-627) and cultured in T cell medium composed of RPMI 1640 (Gibco, cat. no. 11875093), 10% FCS (HyClone, cat. no. SH30071.03), 2 mM L-alanyl-L-glutamine (GlutaMAX; Gibco, cat. no. 35050061), 1 mM sodium pyruvate (Gibco, cat. no. 11360070), 0.1 mM non-essential amino acids (Gibco, cat. no. 11140050), 55 μM 2-mercaptoethanol (Gibco, cat. No. 21985-023), 100 U/ml penicillin (PenStrep; Gibco, cat. no. 15140-122), 100 μg/ml streptomycin (PenStrep; Gibco, cat. no. 15140-122), 10 mM HEPES (Gibco, cat. no. 15630080), along with 5 ng/ml IL-7 and 5 ng/ml IL-15.

Table 1 shows the agents and treatment protocol for the study.

Recipients: C57BL/6J.

Donors: Wild Type: BALB/cJ (BALB); Engineered: IL-12-knock-in BALB/cJ (IL-12-BALB).

Tumor infusion method: sub-cutaneous.

TABLE 1
Agents and Treatment Protocols

| | | 70,000 | | |
|---|---|---|---|---|
| | | TC-1-luc | Cyclophosphamide | 1 Million CD4+ |
| Group | N | S.C. | 200 mg/kg IP d 14 | T Cells IV day 15 |
| 1 | 10 | + | − | — |
| 2 | 10 | + | + | — |
| 3 | 10 | + | + | BALB CD4+ T cells |
| 4 | 10 | + | + | IL-12-BALB CD4+ |
| | | | | T cells |

Mice will be followed for survival and tumor volume. Tumor volume will be measured twice weekly with calipers and volume calculated as V=(L×W2)/2.

### Example 2. Validating Allogeneic Adoptive T Leukocytes in Over-Expression of Inflammatory Cytokines

The objective of this experiment is to validate the treatment benefit resulting from infusion of an adoptive cell therapy composition composed of alloreactive CD4+ T cells engineered to over-express an inflammatory cytokine, Interferon-γ.

This experiment uses the TC-1-Luc lung cancer cell line infused into C57BL/6 mice as a model for human tumors, including lung cancer. Allogeneic BALB/c CD4+ T cells are used as a platform for production and delivery of an excess of the inflammatory cytokine, Interferon-γ.

Allogeneic CD4+ T cells over-expressing Interferon-γ will be generated by knocking an IFNG sequence into the PDCDI locus of BALB/c primary CD4+ T cells (IFNG-BALBs), using high-efficiency non-viral CRISPR/Cas9-mediated gene editing, as described elsewhere. See, Oh et al., (2022), J. Exp. Med., 219(5):e20211530.

CD4+ T cells from donors will be isolated by negative selection using Miltenyi CD4+ T Cell Isolation Kits, mouse (Miltenyi, cat. no. 130-104-454) as per Miltenyi protocol. T cells will be activated 1 day post-nucleofection with Miltenyi T Cell Activation/Expansion kits (cat. no. 130-093-627) and cultured in T cell medium composed of RPMI 1640 (Gibco, cat. no. 11875093), 10% FCS (HyClone, cat. no. SH30071.03), 2 mM L-alanyl-L-glutamine (GlutaMAX; Gibco, cat. no. 35050061), 1 mM sodium pyruvate (Gibco, cat. no. 11360070), 0.1 mM non-essential amino acids (Gibco, cat. no. 11140050), 55 μM 2-mercaptoethanol (Gibco, cat. No. 21985-023), 100 U/ml penicillin (PenStrep; Gibco, cat. no. 15140-122), 100 μg/ml streptomycin (PenStrep; Gibco, cat. no. 15140-122), 10 mM HEPES (Gibco, cat. no. 15630080), along with 5 ng/ml IL-7 and 5 ng/ml IL-15.

Table 2 shows the agents and treatment protocol for the study.

Recipients: C57BL/6J

Donors: Wild Type: BALB/cJ (BALB); Engineered: IFNG-knock-in BALB/cJ (IFNG-BALB)

Tumor infusion method: sub-cutaneous.

TABLE 2
Agents and Treatment Protocols

| | | 70,000 | | |
|---|---|---|---|---|
| | | TC-1-luc | Cyclophosphamide | 1 Million CD4+ |
| Group | N | S.C. | 200 mg/kg IP d 14 | T Cells IV day 15 |
| 1 | 10 | + | − | — |
| 2 | 10 | + | + | — |
| 3 | 10 | + | + | BALB CD4+ T cells |
| 4 | 10 | + | + | IFNG-BALB CD4+ |
| | | | | T cells |

Mice will be followed for survival and tumor volume. Tumor volume will be measured twice weekly with calipers and volume calculated as V=(L×W²)/2.

### Example 3. Validating Allogeneic Adoptive T Leukocytes in Production of Anti-Inflammatory Cytokines

The objective of this experiment is to validate the efficacy of an adoptive cell therapy composition composed of alloreactive CD4+ T cells engineered to express an anti-inflammatory cytokine upon activation, IL-10.

C57BL/6 mouse cell cultures are used as a model for human patients suffering inflammatory disease.

Allogeneic CD4+ T cells expressing Insulin will be generated by knocking IL-10 into the PDCDI locus of BALB/c primary CD4+ T cells (IL10-BALBs), using high-efficiency non-viral CRISPR/Cas9-mediated gene editing, as described elsewhere. See, Oh et al., (2022), J. Exp. Med., 219(5):e20211530. The efficacy of IL-10 production by these cells will be tested in a standard mixed lymphocyte reaction (MLR). IL-10 production will be tested by ELISA.

CD4+ T cells from donors will be isolated by negative selection using Miltenyi CD4+ T Cell Isolation Kits, mouse (Miltenyi, cat. no. 130-104-454) as per Miltenyi protocol. T cells will be activated 1 day post-nucleofection with Miltenyi T Cell Activation/Expansion kits (cat. no. 130-093-627) and cultured in T cell medium composed of RPMI 1640 (Gibco, cat. no. 11875093), 10% FCS (HyClone, cat. no. SH30071.03), 2 mM L-alanyl-L-glutamine (GlutaMAX; Gibco, cat. no. 35050061), 1 mM sodium pyruvate (Gibco, cat. no. 11360070), 0.1 mM non-essential amino acids (Gibco, cat. no. 11140050), 55 μM 2-mercaptoethanol (Gibco, cat. No. 21985-023), 100 U/ml penicillin (PenStrep; Gibco, cat. no. 15140-122), 100 μg/ml streptomycin (PenStrep; Gibco, cat. no. 15140-122), 10 mM HEPES (Gibco, cat. no. 15630080), along with 5 ng/ml IL-7 and 5 ng/ml IL-15. IL-10 production will be quantified with Invitrogen IL-10 Mouse ELISA Kit (Invitrogen, cat. no. BMS614).

Recipient Cell Type: C57BL/6J

Donor Cell Type: Wild Type: BALB/cJ (BALB); Engineered: IL-10-knock-in BALB/cJ (IL10-BALB).

### Example 4. Validating Allogeneic Adoptive T Leukocytes in Production of Non-Wild-Type Molecules (Hormone)

The objective of this experiment is to validate the efficacy of an adoptive cell therapy composition composed of alloreactive CD4+ T cells engineered to express a hormone, insulin.

C57BL/6 mouse cell cultures are used as a model for hormone-deficient human patients.

Allogeneic CD4+ T cells expressing insulin will be generated by knocking Ins1 into the PDCD1 locus of BALB/c primary CD4+ T cells (INS1-BALBs), using high-efficiency non-viral CRISPR/Cas9-mediated gene editing, as described elsewhere. See, Oh et al., (2022), J. Exp. Med., 219(5):e20211530. Insulin production by these cells will be tested in a standard mixed lymphocyte reaction (MLR). Insulin production will be tested by ELISA.

CD4+ T cells from donors will be isolated by negative selection using Miltenyi CD4+ T Cell Isolation Kits, mouse (Miltenyi, cat. no. 130-104-454) as per Miltenyi protocol. T cells will be activated 1 day post-nucleofection with Miltenyi T Cell Activation/Expansion kits (cat. no. 130-093-627) and cultured in T cell medium composed of RPMI 1640 (Gibco, cat. no. 11875093), 10% FCS (HyClone, cat. no. SH30071.03), 2 mM L-alanyl-L-glutamine (GlutaMAX; Gibco, cat. no. 35050061), 1 mM sodium pyruvate (Gibco, cat. no. 11360070), 0.1 mM non-essential amino acids (Gibco, cat. no. 11140050), 55 μM 2-mercaptoethanol (Gibco, cat. No. 21985-023), 100 U/ml penicillin (PenStrep; Gibco, cat. no. 15140-122), 100 μg/ml streptomycin (PenStrep; Gibco, cat. no. 15140-122), 10 mM HEPES (Gibco, cat. no. 15630080), along with 5 ng/ml IL-7 and 5 ng/ml IL-15. Insulin production will be quantified with Invitrogen Mouse INSULIN ELISA Kit (Invitrogen, cat. no. EMINS).

Recipient Cells: C57BL/6.

Donors: Wild Type: BALB/cJ (BALB); Engineered: Ins-1-knock-in BALB/cJ (INS-BALB).

What is claimed is:

1. A pharmaceutical composition comprising a plurality of isolated leukocytes that are obtained from a subject; and wherein the leukocytes are modified to increase the expression of IL-2, FLT3L, IFN gamma, IFN gamma, IFN alpha, IFN beta, IL-12, TNF alpha, IL-1 beta, IL-6, CSF2, IL-4, IL-5, IL-10, IL-13, IL-21, TGF-β, or combinations thereof.

2. A pharmaceutical composition comprising a plurality of isolated leukocytes that are obtained from a donor subject and are mismatched to a recipient subject for at least one human leukocyte antigen (HLA) Class II allele in the donor versus recipient (graft-versus-host) direction relative to the recipient subject,
wherein the leukocytes are depleted of CD8+ T cells by about 10-fold or greater relative to un-depleted leukocytes,
wherein the leukocytes are modified to increase the expression of IL-2, FLT3L, IFN gamma, CSF2, IL-4, IL-5, IL-10, IL-13, IL-21, IFN gamma, IFN alpha, IFN beta, IL-12, TNF alpha, IL-1 beta, IL-6, TGF-β or combinations thereof.

3. A pharmaceutical composition comprising a plurality of isolated leukocytes obtained from an allogeneic donor subject, wherein the donor CD4+ T cells have been stimulated in vivo or ex vivo by an antigen present in a recipient subject, and the donor subject comprises at least one HLA Class II allele match relative to the recipient,
wherein the leukocytes are depleted of CD8+ T cells by about 10-fold or greater relative to un-depleted leukocytes,
wherein the leukocytes are modified to increase the expression of IL-2, FLT3L, IFN gamma, IFN alpha, IFN beta, IL-12, TNF alpha, IL-1 beta, IL-6, IFN gamma, CSF2, IL-4, IL-5, IL-10, IL-13, IL-21, TGF-β or combinations thereof.

4. A pharmaceutical composition comprising a plurality of isolated leukocytes that are obtained from a donor subject and (i) are mismatched to a recipient subject for at least one HLA Class II allele mismatch in the donor versus recipient (graft-versus-host) direction relative to the recipient subject and (ii) the donor CD4+ T cells have been stimulated in vivo or ex vivo by an antigen present in a recipient subject, and the donor subject comprises at least one human leukocyte HLA Class II allele match relative to the recipient,
wherein the leukocytes are depleted of CD8+ T cells by about 10-fold or greater relative to un-depleted leukocytes,
wherein at least a portion of the CD4+ T cells are modified to increase the activity of IL-2, FLT3L, IFN gamma, CSF2, IL-4, IL-5, IL-10, IL-13, IFN gamma, IFN alpha, IFN beta, IL-12, TNF alpha, IL-1 beta, IL-6, IL-21, TGF-β or combinations thereof.

5. The pharmaceutical composition of any one of the preceding claims, wherein at least a portion of the T cells are differentiated to Tₕ1 or T follicular helper (T_{fh}) CD4+ T cells.

6. The pharmaceutical composition of any one of claims 1-5, wherein the T cells are biased toward Tₕ1 CD4+ T cell differentiation by increasing the expression of IL-2, FLT3L, IFN gamma, CSF2, IL-4, IL-5, IL-10, IL-13, IL-21, IFN gamma, IFN alpha, IFN beta, IL-12, TNF alpha, IL-1 beta, IL-6, TGF-β or combinations thereof.

7. The pharmaceutical composition of any one of the preceding claims, wherein expression of IL-2, FLT3L, IFN gamma, CSF2, IL-4, IL-5, IL-10, IFN gamma, IFN alpha, IFN beta, IL-12, TNF alpha, IL-1 beta, IL-6, IL-13, IL-21, TGF-β or combinations thereof is increased with a pharmacological agent or by genetic modification.

8. The pharmaceutical composition of claim 7, wherein expression of IL-2, FLT3L, IFN gamma, CSF2, IL-4, IL-5, IL-10, IL-13, or IL-21 is increased by modifying the IL-2 gene, the FLT3L gene, the IFN gamma gene, the CSF2 gene, the IL-4 gene, the IL-5 gene, the IL-10 gene, the IL-13 gene, the IL-21 gene, the IFN gamma gene, the IFN alpha gene, the IFN beta gene, the IL-12 gene, the TNF alpha gene, the IL-1 beta gene, the IL-6 gene, or the TGF-β gene.

9. The pharmaceutical composition of claim 8, wherein the IL-2 gene, the FLT3L gene, the IFN gamma gene, the CSF2 gene, the IL-4 gene, the IL-5 gene, the IL-10 gene, the IL-13 gene, the IL-21 gene, the IFN gamma gene, the IFN alpha gene, the IFN beta gene, the IL-12 gene, the TNF alpha gene, the IL-1 beta gene, the IL-6 gene, or the TGF-β gene is modified using CRISPR, TALEN, base editing, prime editing, or PASTE.

10. The pharmaceutical composition of any one of claims 1-5, wherein differentiation of the T cells into T regulatory cells is attenuated with a pharmacological agent or by genetic modification.

11. The pharmaceutical composition of any one of claims 1-5, wherein differentiation of the T cells into Tₕ2 cells or function as Tₕ2 cells is attenuated with a pharmacological agent or by genetic modification.

12. The pharmaceutical composition of any one of claims 2-11, wherein the HLA Class II match is an HLA-DRB1 allele, an HLA-DQB1 allele, or an HLA-DPB1 allele.

13. The pharmaceutical composition of any one of the preceding claims, wherein activation of myeloid cells is inhibited.

14. The pharmaceutical composition of any one of claims 1-13, wherein IL-2, FLT3L, IFN gamma, CSF2, IL-4, IL-5, IL-10, IL-13, IL-21, or TGF-β is increased in CD4+ T cells by at least about 50% relative to IL-2, FLT3L, IFN gamma, CSF2, IL-4, IL-5, IFN gamma, IFN alpha, IFN beta, IL-12, TNF alpha, IL-1 beta, IL-6, IL-10, IL-13, IL-21, or TGF-β basal activity, respectively.

15. The pharmaceutical composition of any one of claims 1-13, wherein IL-2 expression is increased in CD4+ T cells by at least about 50% relative to IL-2 basal activity.

16. The pharmaceutical composition of any one of claims 1-15, wherein the amount of IL-2, FLT3L, IFN gamma, CSF2, IL-4, IL-5, IL-10, IL-13, IFN gamma, IFN alpha, IFN beta, IL-12, TNF alpha, IL-1 beta, IL-6, IL-21 or TGF-β is measured by Western Blot.

17. A method for producing a leukocyte composition comprising:
a) obtaining a peripheral blood cell composition from a subject
b) isolating leukocytes from the peripheral blood cell composition, and
c) increasing the expression of IL-2, FLT3L, IFN gamma, CSF2, IL-4, IFN gamma, IFN alpha, IFN beta, IL-12, TNF alpha, IL-1 beta, IL-6, IL-5, IL-10, IL-13, IL-21, or TGF-β or combinations thereof.

18. The method of claim 17, further comprising a treatment to promote differentiation of at least a portion of T cells toward Tₕ1 or T_{fh} CD4+ T cells, or to maintain CD4+ Tₕ1 or T_{fh} cells in their state of differentiation.

19. The method of any one of claim 17 or 18, wherein the method further comprises culturing the leukocytes in vitro.

20. The method of any one of claim 17 or 18, wherein the method further comprises culturing the leukocytes ex vivo.

21. The method of any one of claims 17-20, further comprising adding one or more additional cytokines.

22. The method of claim 21, wherein the one or more additional cytokines is IL-2, IL-7, IL-12, IL-15, IL-18, IFNγ, IFN gamma, IFN alpha, IFN beta, IL-12, TNF alpha, IL-1 beta, IL-6, IL-21, or TGF-β.

23. The method of any one of claims 17-22, further comprising adding one or more antibodies.

24. The method of claim 23, wherein the one or more antibodies is an anti-IL-3 antibody, an anti-IL-4 antibody, an anti-CD3 antibody, an anti-CD200 antibody or an anti-CD28 antibody.

25. The method of any one of claims 17-24, wherein the T cells are biased toward Tₕ1 CD4+ T cell differentiation or function by increasing the expression of IL-2, FLT3L, IFN gamma, CSF2, IL-4, IL-5, IL-10, IL-13, IFN gamma, IFN alpha, IFN beta, IL-12, TNF alpha, IL-1 beta, IL-6, IL-21 TGF-β, or combinations thereof.

26. The method of any one of claims 16-24, wherein the T cells are biased toward Tₕ1 CD4+ T cell differentiation or function by increasing the expression of IL-2, FLT3L, IFN gamma, CSF2, IL-4, IL-5, IL-10, IFN gamma, IFN alpha, IFN beta, IL-12, TNF alpha, IL-1 beta, IL-6, IL-13, IL-21, TGF-β or combinations thereof.

27. The method of any one of claims 16-24, wherein the expression of IL-2, FLT3L, IFN gamma, CSF2, IL-4, IL-5, IL-10, IL-13, IFN gamma, IFN alpha, IFN beta, IL-12, TNF alpha, IL-1 beta, IL-6, IL-21, TGF-β or combinations thereof is increased with a pharmacological agent or by genetic modification.

28. The method of any one of claims 16-24, wherein the expression of IL-2, FLT3L, IFN gamma, CSF2, IL-4, IL-5, IL-10, IL-13, IL-21, or TGF-β is increased by modifying the IL-2 gene, the FLT3L gene, the IFN gamma gene, the CSF2 gene, the IL-4 gene, the IL-5 gene, the IL-10 gene, the IL-13 gene, the IL-21 gene, the TGF-β gene, the IFN gamma gene, the IFN alpha gene, the IFN beta gene, the IL-12 gene, the TNF alpha gene, the IL-1 beta gene, the IL-6 gene, or combinations thereof.

29. The method of claim 28, wherein the IL-2 gene, the FLT3L gene, the IFN gamma gene, the CSF2 gene, the IL-4 gene, the IL-5 gene, the IL-10 gene, the IL-13 gene, the IL-21 gene, the TGF-β gene or combinations thereof is modified using CRISPR TALEN, base editing, prime editing, or PASTE.

30. The method of any one of claims 17-29, wherein the HLA Class II allele mismatch is an HLA-DRB1 allele, an HLA-DQB1 allele, or an HLA-DPB1 allele in the donor-versus-recipient (graft-versus-host) direction.

31. The method of any one of claims 17-29, wherein the HLA Class II match is an HLA-DRB1 allele, an HLA-DQB1 allele, or an HLA-DPB1 allele.

32. The method of any one of claims 17-31, wherein activation of myeloid cells is inhibited.

33. The method of any one of claims 17-31, wherein expression of IL-2, FLT3L, IFN gamma, CSF2, IL-4, IL-5, IL-10, IFN gamma, IFN alpha, IFN beta, IL-12, TNF alpha, IL-1 beta, IL-6, IL-13, or IL-21 is increased in CD4+ T cells by at least about 50% relative to IL-2, FLT3L, IFN gamma, CSF2, IL-4, IL-5, IL-10, IL-13, IFN gamma, IFN alpha, IFN beta, IL-12, TNF alpha, IL-1 beta, IL-6, IL-21 basal activity, respectively.

34. The method of any one of claims 17-31, wherein expression of IL-2 is increased in CD4+ T cells by at least about 50% relative to IL-2 basal activity.

35. The method of claim 34, wherein an amount of IL-2, FLT3L, IFN gamma, IFN gamma, IFN alpha, IFN beta, IL-12, TNF alpha, IL-1 beta, IL-6, CSF2, IL-4, IL-5, IL-10, IL-13, IL-21, or TGF-β is measured by Western Blot.

36. A method of treating cancer, comprising administering to a subject in need thereof an effective amount of (i) a lymphodepleting agent and (ii) the pharmaceutical composition of any one of claims 1-16.

37. A method of treating cancer, comprising administering to a subject in need thereof an effective amount of (i) a lymphodepleting agent, (ii) an inhibitor of NLR family pyrin domain containing 3 (NLRP3), and (iii) the pharmaceutical composition of any one of claims 1-16.

38. A method of treating cancer, comprising administering to a subject in need thereof an effective amount of (i) a lymphodepleting agent, (ii) an agent that inhibits differentiation of the T cells into T regulatory cells or inhibits the function of T regulatory cells, and (iii) the pharmaceutical composition of any one of claims 1-16.

39. A method of potentiating anti-tumor immunity in a subject having a cancer, comprising administering to a subject in need thereof an effective amount of (i) a lymphodepleting agent, and (ii) the pharmaceutical composition of any one of claims 1-16.

40. The method of any one of claims 36-39, wherein the lymphodepleting agent is a cytoreductive agent.

41. The method of claim 40, wherein the cytoreductive agent is an alkylating agent, an alkyl sulphonate, a nitrosourea, a triazene, an antimetabolite, a pyrimidine analog, a purine analog, a vinca alkaloids a epipodophyllotoxin, an antibiotic, a dibromomannitol, a deoxyspergualine, a dimethyl myleran or a thiotepa.

42. The method of claim 41, wherein the alkylating agent is cyclophosphamide.

43. The method of claim 41, wherein the purine analog is fludarabine, cladribine, or pentostatin.

44. The method of any one of claims 36-43, wherein the cancer is a hematological cancer.

45. The method of any one of claims 36-43, wherein the cancer is a solid cancer.

46. The method of claim 44, wherein the hematological cancer is a leukemia, lymphoma, multiple myeloma, myelodysplastic syndrome, or myeloproliferative disorder.

47. The method of claim 46, wherein the leukemia, lymphoma, multiple myeloma, myelodysplastic syndrome or myeloproliferative disorder is non-Hodgkin lymphoma, chronic lymphocytic leukemia, small lymphocytic lymphoma, diffuse large B-cell lymphoma, follicular lymphoma, marginal zone lymphoma, mantle cell lymphoma, acute lymphoblastic leukemia, acute myeloid leukemia, hairy cell leukemia, AIDS-related lymphoma, cutaneous T cell lymphoma, Hodgkin lymphoma, mycosis fungoides, primary central nervous system lymphoma, Sezary syndrome, T cell lymphoma, Waldenström's macroglobulinemia, chronic myeloid leukemia, chronic myelomonocytic leukemia, polycythemia vera, essential thrombocythemia, or idiopathic myelofibrosis.

48. The method of claim 45, wherein the solid cancer is sarcoma, carcinoma, a neurofibromatoma, a colon cancer, a lung cancer, an ovarian cancer, a pancreatic cancer, or a breast cancer.

49. The method of any one of claims 36-48, further comprising administering to the subject in need thereof an additional therapeutic agent.

50. The method of claim 49, wherein the additional therapeutic agent is a chemotherapeutic agent, radiation therapy, an immunotherapeutic agent, a T cell agonist cytokine, a CAR-T, a CAR-NK, a bispecific or trispecific T cell or NK cell engager, natural killer cells, gamma-delta T cells, antibody-drug conjugate, an antibody, an immune checkpoint inhibitor, small molecule inhibitor, or an oncolytic virus therapy.

51. The method of claim 50, wherein the antibody is rituximab, Obinutuzumab, ofatumumab, cetuximab, trastuzumab, pertuzumab, brentuximab vedotin, gemtuzumab, trastuzumab emtansine, inotuzumab ozogamicin, glembatumumab vedotin, lorvotuzumab mertansine, cantuzumab mertansine, or milatuzumab-doxorubicin.

52. The method of claim 50, wherein the immune checkpoint inhibitor is an inhibitor of or antibody against PD-L1, PD-1, CTLA-4, LAG-3, TIGIT, or TIM-3.

53. The method of claim 50, wherein the small molecule inhibitor is dasatinib, nilotinib, ponatinib, imatinib, bosutinib, asciminib, lapatinib, or vismodegib.

54. The method of any one of claims 50-53, wherein the pharmaceutical composition is administered after the lymphodepleting agent.
